(19) United States
(12) Patent Application Publication
(10) Pub. No.: US 2023/0374463 A1
(43) Pub. Date: Nov. 23, 2023

(54) DEGENERATING OVARIAN MICROENVIRONMENT RESISTANT MESENCHYMAL STEM CELLS

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Courtney BARTLETT, Niceville, FL (US)

(73) Assignee: CREATIVE MEDICAL TECHNOLOGIES, INC., Phoenix, AZ (US)

(21) Appl. No.: 18/311,768

(22) Filed: May 3, 2023

Related U.S. Application Data
(60) Provisional application No. 63/343,841, filed on May 19, 2022.

(51) Int. Cl. C12N 5/0775

(52) U.S. Cl. CPC C12N 5/0662; C12N 2506/45; C12N 2500/02

(57) ABSTRACT

Disclosed are compositions of matter, protocols, and procedures useful for generation of mesenchymal stem cells capable of withstanding the microenvironment of degenerating ovaries. In one embodiment mesenchymal stem cells are pre-treated under conditions capable of upregulating cytoprotective responses and genes, such as HIF-1 alpha and hemoxygenase. In other embodiments mesenchymal stem cells are temporarily pulsed with hypoxia and/or acidity in order to prime them for the harsh environment of degenerating ovarian tissue. Through this approach increased viability of mesenchymal stem cell subsequent to intra-ovarian administration is achieved.

### CROSS-REFERENCE TO RELATED APPLICATIONS

This application claims priority to U.S. Provisional Application No. 63/343,841, titled “Degenerating Ovarian Microenvironment Resistant Mesenchymal Stem Cells” filed May 19, 2022, which is hereby incorporated by reference herein in its entirety.

### FIELD OF THE INVENTION

The invention is directed to methods of treating ovarian failure by administering regenerative cells to a patient in need.

### BACKGROUND OF THE INVENTION

Ovarian failure is associated with natural aging. Premature ovarian failure (POF), also known as primary ovarian insufficiency, refers to a loss of normal ovarian function before age 40. In a normal karyotype female, POF is characterized by amenorrhea and infertility, with elevated serum levels of follicle-stimulating hormone (FSH) and decreased levels of estrogen. Currently, there is no effective treatment for this condition, and alternatives such as the use of egg donations are prohibitively expensive and ethically unacceptable to some couples. The current invention provides means of stimulating ovarian regeneration by administration of mesenchymal stem cells specifically treated in a manner to enhance their ability to grow and provide therapeutic activity in a degenerative environment.

### SUMMARY

Preferred embodiments include methods of treating ovarian failure comprising administration of a mesenchymal stem cell population generated in a manner to withstand degenerative microenvironments associated with ovarian failure.

Preferred methods include embodiments wherein said degenerative microenvironments associated with ovarian failure are characterized by enhanced neutrophilic infiltration as compared to microenvironment of a non-degenerative ovary.

Preferred methods include embodiments wherein said degenerative microenvironments associated with ovarian failure are characterized by enhanced monocytic infiltration as compared to microenvironment of a non-degenerative ovary.

Preferred methods include embodiments wherein said degenerative microenvironments associated with ovarian failure are characterized by enhanced T cell infiltration as compared to microenvironment of a non-degenerative ovary.

Preferred methods include embodiments wherein said degenerative microenvironments associated with ovarian failure are characterized by enhanced B cell infiltration as compared to microenvironment of a non-degenerative ovary.

Preferred methods include embodiments wherein said degenerative microenvironments associated with ovarian failure are characterized by enhanced NK cell infiltration as compared to microenvironment of a non-degenerative ovary.

Preferred methods include embodiments wherein said degenerative microenvironments associated with ovarian failure are characterized by enhanced NKT cell infiltration as compared to microenvironment of a non-degenerative ovary.

Preferred methods include embodiments wherein said degenerative microenvironments associated with ovarian failure are characterized by enhanced gamma delta cell infiltration as compared to microenvironment of a non-degenerative ovary.

Preferred methods include embodiments wherein said degenerative microenvironments associated with ovarian failure are characterized by enhanced fibrotic cell infiltration as compared to microenvironment of a non-degenerative ovary.

Preferred methods include embodiments wherein said degenerative microenvironments associated with ovarian failure are characterized by upregulated production of interferon gamma as compared to microenvironment of a non-degenerative ovary.

Preferred methods include embodiments wherein said degenerative microenvironments associated with ovarian failure are characterized by upregulated production of interleukin-17 as compared to microenvironment of a non-degenerative ovary.

Preferred methods include embodiments wherein said degenerative microenvironments associated with ovarian failure are characterized by upregulated production of interleukin-18 as compared to microenvironment of a non-degenerative ovary.

Preferred methods include embodiments wherein said degenerative microenvironments associated with ovarian failure are characterized by upregulated production of interleukin-27 as compared to microenvironment of a non-degenerative ovary.

Preferred methods include embodiments wherein said mesenchymal stem cell population is cultured under conditions that endow protection from microenvironment of degenerating ovary.

Preferred methods include embodiments wherein said culture conditions are hypoxic conditions.

Preferred methods include embodiments wherein said hypoxic conditions are conditions of reduced oxygen tension compared to normoxia.

Preferred methods include embodiments wherein hypoxia reference to oxygen is below 20%.

Preferred methods include embodiments wherein hypoxia reference to oxygen is below 15%.

Preferred methods include embodiments wherein hypoxia reference to oxygen is approximately 1-10%.

Preferred methods include embodiments wherein said cells are generated by introduction into cytoplasm of said mesenchymal stem cell extracts of cytoplasm from immune modulatory antigen presenting cells and/or defined transcription factors.

Preferred methods include embodiments wherein said ability to withstand degenerative microenvironments associated with ovarian failure is ability to suppress proliferation of responding lymphocytes in a mixed lymphocyte reaction.

Preferred methods include embodiments wherein said ability to withstand degenerative microenvironments associated with ovarian failure is ability of said mesenchymal stem cells to suppress Th1 cytokine production of responding lymphocytes in a mixed lymphocyte reaction.

Preferred methods include embodiments wherein said Th1 cytokines are one or more cytokine selected from a group comprising of: a) IL-2; b) IL-12; c) IL-15; d) IL-18; and e) interferon gamma.

Preferred methods include embodiments wherein said ability to withstand degenerative microenvironments associated with ovarian failure is ability of said mesenchymal stem cells to suppress proliferative response of a T cell in response to a proliferative signal.

Preferred methods include embodiments wherein said proliferative signal is a mitogen.

Preferred methods include embodiments wherein said proliferative signal is a crosslinking antibody.

Preferred methods include embodiments wherein said proliferative signal is a cytokine.

Preferred methods include embodiments wherein said mitogen is selected from a group comprising of: a) phytohemagglutinin; b) pokeween mitogen; and c) conconavalin A.

Preferred methods include embodiments wherein said antibody crosslinks molecules selected from a group comprising of: a) CD3; b) CD2 and c) CD28.

Preferred methods include embodiments wherein said to ability withstand degenerative microenvironments associated with ovarian failure is ability of cells to induce T regulatory cells.

Preferred methods include embodiments wherein said T regulatory cells possess membrane bound TGF-beta.

Preferred methods include embodiments wherein said T regulatory cells inhibit proliferation of T cells.

Preferred methods include embodiments wherein said T regulatory cells inhibit cytokine production by T cells.

Preferred methods include embodiments wherein ability to withstand degenerative microenvironments associated with ovarian failure is ability to inhibit dendritic cell maturation.

Preferred methods include embodiments wherein said dendritic cell maturation is augmentation of expression of costimulatory molecules.

Preferred methods include embodiments wherein said dendritic cell maturation is inhibition of expression of coinhibitory molecules.

Preferred methods include embodiments wherein said cells are derived by isolation from a placenta.

Preferred methods include embodiments wherein stem cells are extracted from said placenta, by the steps of: dissociating fetal vascular lobules from a hemochorial placenta; digesting the dissociated fetal vascular lobes with an enzymatic mixture or by mechanical means; applying a filtration means to said dissociated lobes in order to remove particulates; obtaining mononuclear cells; plating said mononuclear cells in a substrate allowing for growth of said mononuclear cells to confluency; detaching the confluent cells from the plate; and isolating for expression of CD144 and substantially lack of expression of CD45.

Preferred methods include embodiments wherein dissociation of fetal vascular lobes is accomplishing by incubation with a mixture of about 2% collagenase, about 0.25% trypsin and about 0.1% DNAse in tissue culture medium.

Preferred methods include embodiments wherein dissociation of fetal vascular lobes is accomplishing by incubation with a mixture of about 2% collagenase, about 0.25% trypsin and about 0.1% DNAse in tissue culture medium.

Preferred methods include embodiments wherein cells isolated are comprised of adherent cells expressing the marker CD73 but substantially lacking CD105.

Preferred methods include embodiments wherein cells isolated are comprised of adherent cells expressing the marker CD73 and CD105 but lacking in CD90.

Preferred methods include embodiments wherein said regenerative adjuvant is an anti-inflammatory cytokine.

Preferred methods include embodiments wherein said anti-inflammatory cytokine is selected from a group comprising of IL-4.

Preferred methods include embodiments wherein said anti-inflammatory cytokine is selected from a group comprising of PGE-2.

Preferred methods include embodiments wherein said anti-inflammatory cytokine is selected from a group comprising of IL-1 receptor antagonist.

Preferred methods include embodiments wherein said anti-inflammatory cytokine is selected from a group comprising of IL-10.

Preferred methods include embodiments wherein said anti-inflammatory cytokine is selected from a group comprising of IL-13.

Preferred methods include embodiments wherein said anti-inflammatory cytokine is selected from a group comprising of IL-14.

Preferred methods include embodiments wherein said anti-inflammatory cytokine is selected from a group comprising of IL-20.

Preferred methods include embodiments wherein said anti-inflammatory cytokine is selected from a group comprising of IL-22.

Preferred methods include embodiments wherein said anti-inflammatory cytokine is selected from a group comprising of IL-35.

Preferred methods include embodiments wherein said composition is composed of mesenchymal stem cells, and wherein said first regenerative adjuvant is hypoxia.

Preferred methods include embodiments wherein said mesenchymal stem cells possess one or more markers selected from a group comprising of: a) CD11b; b) CD11c; c) CD20; d) CD56; e) CD57 f) CD73; g) CD90; h) CD105; i) membrane bound TGF-beta; and j) neuropilin.

Preferred methods include embodiments wherein said composition is capable of inhibiting T cell mediated immune responses.

Preferred methods include embodiments wherein said T cell mediated immune responses comprise of Th1 cell production of cytokines.

Preferred methods include embodiments wherein said cytokine is selected from a group of cytokines comprising of: a) IL-2; b) IL-6; c) IL-8; d) IL-12; e) IL-15; f) IL-18; g) interferon gamma; h) TNF-alpha; and i) interleukin-33.

Preferred methods include embodiments wherein said T cell mediated immune responses is activation of gamma delta T cells.

Preferred methods include embodiments wherein said gamma delta T cell activation is production of granzyme.

Preferred methods include embodiments wherein said gamma delta T cell activation is production of perforin.

Preferred methods include embodiments wherein said gamma delta T cell activation is production of interferon gamma.

Preferred methods include embodiments wherein said T cell mediated immune response is proliferation of a T cell.

Preferred methods include embodiments wherein said T cell mediated immune response is activation of a CD8 T cell.

Preferred methods include embodiments wherein said activation of CD8 cell is proliferation of said CD8 cell.

Preferred methods include embodiments wherein said activation of CD8 cell is production of perforin.

Preferred methods include embodiments wherein said activation of CD8 cell is production of granzyme.

Preferred methods include embodiments wherein said activation of CD8 cells is induction of cytotoxicity.

Preferred methods include embodiments wherein said activation of CD8 cells is production of inflammatory cytokines.

Preferred methods include embodiments wherein said inflammatory cytokine is RANTES.

Preferred methods include embodiments wherein said inflammatory cytokine is MIP-1 alpha.

Preferred methods include embodiments wherein said inflammatory cytokine is MIP-1 beta.

Preferred methods include embodiments wherein said inflammatory cytokine is IL-2.

Preferred methods include embodiments wherein said inflammatory cytokine is IL-6.

Preferred methods include embodiments wherein said inflammatory cytokine is IL-8.

Preferred methods include embodiments wherein said inflammatory cytokine is IL-12.

Preferred methods include embodiments wherein said inflammatory cytokine is IL-15.

Preferred methods include embodiments wherein said inflammatory cytokine is IL-18.

Preferred methods include embodiments wherein said inflammatory cytokine is IL-17.

Preferred methods include embodiments where the mesenchymal cell is derived from an induced pluripotent stem cell (iPSc).

### DESCRIPTION OF THE INVENTION

The invention provides means of generating a type of resilient mesenchymal stem cell useful for treatment of ovarian failure. In one embodiment the invention provides novel stem cell types, methods of manufacture, and therapeutic uses. Provided are means of deriving stem cells possessing regenerative, immune modulatory, anti-inflammatory, and angiogenic/neurogenic activity from umbilical cord tissue such as Umbilical cord tissue. In some embodiments manipulation of stem cell “potency” is disclosed through hypoxic manipulation, growth on non-xenogeneic conditions, as well as addition of epigenetic modulators.

In one embodiment, the cells of the invention are cultured under hypoxia, in one embodiment, cultured in order to induce and/or augment expression of chemokine receptors. One such receptor is CXCR-4. The population of cells, including population of umbilical cord mesenchymal cells, may be enriched for CXCR-4, such as (or such as about) 50%, 55%, 60%, 65%, 70%, 75%, 80%, 85%, 90%, 95%, 96%, 97%, 98%, 99%, or more of the population expressing CXCR-4, CD31, CD34, or any combination thereof. In addition or alternatively, <1%, <2%, <3%, <4%, <5%, <6%, <7%, <8%, <9%, or <10% of the population of cells may express CD14 and/or CD45. The umbilical cord cells of the invention may further possess markers selected from the group consisting of STRO-1, CD105, CD54, CD56, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1, and a combination thereof. In some embodiments said placental cells of the invention are admixed with endothelial cells. Said endothelial cells may express one or more markers selected from the group consisting of: a) extracellular vimentin; b) CD133; c) c-kit; d) VEGF receptor; e) activated protein C receptor; and f) a combination thereof. In some embodiments, the population of endothelial cells comprises endothelial progenitor cells.

The population of cells may be allogeneic, autologous, or xenogenic to an individual, including an individual being administered the population of cells. In some embodiments, the population of cells are matched by mixed lymphocyte reaction matching.

In some embodiments, the population of cells is derived from tissue selected from the group consisting of the placental body, placenta, umbilical cord tissue, peripheral blood, hair follicle, cord blood, Umbilical cord tissue, menstrual blood, endometrium, skin, omentum, amniotic fluid, induced pluripotent stem cell and a combination thereof. In some embodiments, the population of cells, the population of umbilical mesenchymal stem cells, or the population of endothelial cells comprises human umbilical cord derived adherent cells. The human umbilical cord derived adherent cells may express a cytokines selected from the group consisting of) FGF-1; b) FGF-2; c) HGF; d) interleukin-1 receptor antagonist; and e) a combination thereof. In some embodiments, the population of cells, the population of umbilical cord cells express arginase, indoleamine 2,3 deoxygenase, interleukin-10, and/or interleukin 35. In some embodiments, the population of cells, the population of umbilical cord cells, or the population of endothelial cells express hTERT and Oct-4 but does not express a STRO-1 marker.

In some embodiments, a population of stem cells are utilized to induce regeneration of damaged ovarian tissue. In some embodiments, administration of “resilient” mesenchymal stem cells, which have been pulsed by various means such as hypoxia, is associated with endowment of expression of the initerleukin-3 receptor on ovarian tissue. For the practice of the invention, MSC are used to induce dedifferentiation or rejuvenation of ovarian tissues. “Mesenchymal stem cell” or “MSC” in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or ore mesenchymal stem cell can be used interchangeably. Said MSC can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs). the population of umbilical cord cells has an ability to undergo cell division in less than 36 hours in a growth medium. In some embodiments, the population of cells, the population of umbilical cord cells has an ability to proliferate at a rate of 0.9-1.2 doublings per 36 hours in growth media. In some embodiments, the population of cells, the population of umbilical cord cells has an ability to proliferate at a rate of 0.9, 1.0, 1.1, or 1.2 doublings per 36 hours in growth media. The population of cells, population of umbilical cord cells may produce exosomes capable of inducing more than 50% proliferation when the exosomes are cultured with human umbilical cord endothelial cells. The induction of proliferation may occur when the exosomes are cultured with the human umbilical cord endothelial cells at a concentration of 10, 20, 30, 40, 50, 60, 70, 80, 90, 100, 110, 120, 130, 140, 150, 160, 170, 180, 190, 200, or more exosomes per cell.

In some embodiments, a population of cells, including a population of umbilical cells alone, are administered to an individual, including an individual having and acute or chronic pathology, wherein the population of cells may be administered via any suitable route, including as non-limiting examples, intramuscularly and/or intravenously.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

Chemical Modification: As used herein, “chemical modification” refers to the process wherein a chemical or biochemical is used to induce genomic changes in the donor cell, or nucleus thereof, that allow the donor cell, or nucleus thereof, to be responsive during maturation and receptive to the host cell cytoplasm.

Committed: As used herein, “committed” refers to cells which are considered to be permanently committed to a specific function. Committed cells are also referred to as “terminally differentiated cells.”

Cytoplast Extract Modification: As used herein, “cytoplast extract modification” refers to the process wherein a cellular extract consisting of the cytoplasmic contents of a cell are used to induce genomic changes in the donor cell, or nucleus thereof, that allow the donor cell, or nucleus thereof, to be responsive during maturation and receptive to the host cell cytoplasm.

Dedifferentiation: As used herein, “dedifferentiation” refers to loss of specialization in form or function. In cells, dedifferentiation leads to an a less committed cell.

Differentiation: As used herein, “differentiation” refers to the adaptation of cells for a particular form or function. In cells, differentiation leads to a more committed cell.

Donor Cell: As used herein, “donor cell” refers to any diploid (2N) cell derived from a pre-embryonic, embryonic, fetal, or post-natal multi-cellular organism or a primordial sex cell which contributes its nuclear genetic material to the hybrid stem cell. The donor cell is not limited to those cells that are terminally differentiated or cells in the process of differentiation. For the purposes of this invention, donor cell refers to both the entire cell or the nucleus alone.

Donor Cell Preparation: As used herein, “donor cell preparation” refers to the process wherein the donor cell, or nucleus thereof, is prepared to undergo maturation or prepared to be receptive to a host cell cytoplasm and/or responsive within a post-natal environment.

Germ Cell: As used herein, “germ cell” refers to a reproductive cell such as a spermatocyte or an oocyte, or a cell that will develop into a reproductive cell.

Host Cell: As used herein, “host cell” refers to any multipotent stem cell derived from a pre-embryonic, embryonic, fetal, or post-natal multicellular organism that contributes the cytoplasm to a hybrid stem cell.

Host Cell Preparation: As used herein, “host cell preparation” refers to the process wherein the host cell is enucleated.

Hybrid Stem Cell: As used herein, “hybrid stem cell” refers to any cell that is multipotent and is derived from an enucleated host cell and a donor cell, or nucleus thereof, of a multicellular organism. Hybrid stem cells are further disclosed in co-pending U.S. patent application Ser. No. 10/864,788.

Karyoplast Extract Modification: As used herein, “karyoplast extract modification” refers to the process wherein a cellular extract consisting of the nuclear contents of a cell, lacking the DNA, are used to induce genomic changes in the donor cell, or nucleus thereof, that allow the donor cell, or nucleus thereof, to be responsive during maturation or receptive to the host cell cytoplasm.

Maturation: As used herein, “maturation” refers to a process of coordinated steps either forward or backward in the differentiation pathway and can refer to both differentiation or de-differentiation. As used herein, maturation is synonymous with the terms develop or development when applied to the process described herein.

Modified Germ Cell: As used herein, “modified germ cell” refers to a cell comprised of a host enucleated ovum and a donor nucleus from a spermatogonia, oogonia or a primordial sex cell. The host enucleated ovum and donor nucleus can be from the same or different species. A modified germ cell can also be called a “hybrid germ cell.”

Multipotent Adult Progenitor Cells: As used herein, “multipotent adult progenitor cells” refers to multipotent cells isolated from the bone marrow which have the potential to differentiate into mesenchymal, endothelial and endodermal lineage cells.

Pre-embryo: As used herein, “pre-embryo” refers to a fertilized egg in the early stage of development prior to cell division. During the pre-embryonic stage the initial stages of cleavage are occurring.

Pre-embryonic Stem Cell: See “Embryonic Stem Cell” above.

Post-natal Stem Cell: As used herein, “post-natal stem cell” refers to any cell that is multipotent and derived from a multi-cellular organism after birth.

Pluripotent: As used herein, “pluripotent” refers to cells that can give rise to any cell type except the cells of the placenta or other supporting cells of the uterus.

Primordial Sex Cell: As used herein, “primordial sex cell” refers to any diploid cell that is derived from the male or female mature or developing gonad, is able to generate cells that propagate a species and contains a diploid genomic state. Primordial sex cells can be quiescent or actively dividing. These cells include male gonocytes, female gonocytes, spermatogonial stem cells, ovarian stem cells, oogonia, type-A spermatogonia, Type-B spermatogonia. Also known as germ-line stem cells.

Primordial Germ Cell: As used herein, “primordial germ cell” refers to cells present in early embryogenesis that are destined to become germ cells.

Reprogamming: As used herein “reprogramming” refers to the resetting of the genetic program of a cell such that the cell exhibits pluripotency and has the potential to produce a fully developed organism.

Responsive: As used herein, “responsive” refers to the condition of a cell, or group of cells, wherein they are susceptible to and can function accordingly within a cellular environment. Responsive cells are capable of responding to and functioning in a particular cellular environment, tissue, organ and/or organ system.

Somatic Stem Cells: As used herein, “somatic stem cells” refers to diploid multipotent or pluripotent stem cells. Somatic stem cells are not totipotent stem cells. Stem cells are primitive cells that give rise to other types of cells. Also called progenitor cells, there are several kinds of stem cells. Totipotent cells are considered the “master” cells of the body because they contain all the genetic information needed to create all the cells of the body plus the placenta, which nourishes the human embryo. Human cells have this totipotent capacity only during the first few divisions of a fertilized egg. After three to four divisions of totipotent cells, there follows a series of stages in which the cells become increasingly specialized. The next stage of division results in pluripotent cells, which are highly versatile and can give rise to any cell type except the cells of the placenta or other supporting tissues of the uterus. At the next stage, cells become multipotent, meaning they can give rise to several other cell types, but those types are limited in number. An example of multipotent cells is hematopoietic cells—blood cells that can develop into several types of blood cells, but cannot develop into brain cells. At the end of the long chain of cell divisions that make up the embryo are “terminally differentiated” cells—cells that are considered to be permanently committed to a specific function.

Therapeutic Cloning: As used herein, “therapeutic cloning” refers to the cloning of cells using nuclear transfer methods including replacing the nucleus of an ovum with the nucleus of another cell and stem cells derived from the inner cell mass.

Therapeutic Reprogramming: As used herein, “therapeutic reprogramming” refers to the process of maturation wherein a stem cell is exposed to stimulatory factors according to the teachings of the present invention to yield either pluripotent, multipotent or tissue-specific committed cells. Therapeutically reprogrammed cells are useful for implantation into a host to replace or repair diseased, damaged, defective or genetically impaired tissue. The therapeutically reprogrammed cells of the present invention do not possess non-human sialic acid residues.

Totipotent: As used herein, “totipotent” refers to cells that contain all the genetic information needed to create all the cells of the body plus the placenta. Human cells have the capacity to be totipotent only during the first few divisions of a fertilized egg.

Whole Cell Extract Modification: As used herein, “whole cell extract modification” refers to the process wherein a cellular extract consisting of the cytoplasmic and nuclear contents of a cell are used to induce genomic changes in the donor cell, or nucleus thereof, that allow the donor cell, or nucleus thereof, to be responsive during maturation and receptive to the host cell cytoplasm.

In one embodiment the invention teaches phenotypically defined MSC which can be isolated from the Umbilical cord tissue of umbilical cord segments and defined morphologically and by cell surface markers. By dissecting out the veins and arteries of cord segments and exposing the Umbilical cord tissue, the cells of invention, of one embodiment of the invention, may be obtained. An approximately 1-5 cm cord segment is placed in collagenase solution (1 mg/ml, Sigma) for approximately 18 hrs at room temperature. After incubation, the remaining tissue is removed and the cell suspension is diluted with PBS into two 50 ml tubes and centrifuged. Cells are then washed in PBS and counted using hematocytometer. 5-20.times.10.sup.6 cells were then plated in a 6 cm tissue culture plate in low-glucose DMEM (Gibco) with 10% FBS (Hyclone), 2 mM L-Glutamine (Gibco), 100 U/ml penicillin/100 ug/ml streptomycin/0.025 ug/ml amphotericin B (Gibco). At this step of the purification process, cells are exposed to hypoxia. The amount of hypoxia needed is the sufficient amount to induce activagion of HIF-1 alpha. In one embodiment cells are cultured for 24 hours at 2% oxygen. After 48 hrs cells are washed with PBS and given fresh media. Cells were given new media twice weekly. After 7 days, cells are approximately 70-80% confluent and are passed using HyQTase (Hyclone) into a 10 cm plate. Cells are then regularly passed 1:2 every 7 days or upon reaching 80% confluence.

In another embodiment of the invention, biologically useful stem cells are disclosed, of the mesenchymal or related lineages, which are therapeutically reprogrammed cells having minimal oxidative damage and telomere lengths that compare favorably with the telomere lengths of undamaged, pre-natal or embryonic stem cells (that is, the therapeutically reprogrammed cells of the present invention possess near prime physiological state genomes). Moreover the therapeutically reprogrammed cells of the present invention are immunologically privileged and therefore suitable for therapeutic applications. Additional methods of the present invention provide for the generation of hybrid stem cells. Furthermore, the present invention includes related methods for maturing stem cells made in accordance with the teachings of the present invention into specific host tissues. For use in the current invention, the practitioner is thought that ontogeny of mammalian development provides a central role for stem cells. Early in embryogenesis, cells from the proximal epiblast destined to become germ cells (primordial germ cells) migrate along the genital ridge. These cells express high levels of alkaline phosphatase as well as expressing the transcription factor Oct4. Upon migration and colonization of the genital ridge, the primordial germ cells undergo differentiation into male or female germ cell precursors (primordial sex cells). For the purpose of this invention disclosure, only male primordial sex cells (PSC) will be discussed, but the qualities and properties of male and female primordial sex cells are equivalent and no limitations are implied. During male primordial sex cell development, the primordial stem cells become closely associated with precursor sertoli cells leading to the beginning of the formation of the seminiferous cords. When the primordial germ cells are enclosed in the seminiferous cords, they differentiate into gonocytes that are mitotically quiescent. These gonocytes divide for a few days followed by arrest at G0/G1 phase of the cell cycle. In mice and rats these gonocytes resume division within a few days after birth to generate spermatogonial stem cells and eventually undergo differentiation and meiosis related to spermatogenesis. It is known that embryonic stem cells are cells derived from the inner cell mass of the pre-implantation blastocyst-stage embryo and have the greatest differentiation potential, being capable of giving rise to cells found in all three germ layers of the embryo proper. From a practical standpoint, embryonic stem cells are an artifact of cell culture since, in their natural epiblast environment, they only exist transiently during embryogenesis. Manipulation of embryonic stem cells in vitro has lead to the generation and differentiation of a wide range of cell types, including cardiomyocytes, hematopoietic cells, endothelial cells, nerves, skeletal muscle, chondrocytes, adipocytes, liver and pancreatic islets. Growing embryonic stem cells in co-culture with mature cells can influence and initiate the differentiation of the embryonic stem cells to a particular lineage. Maturation is a process of coordinated steps either forward or backward in the differentiation pathway and can refer to both differentiation and/or dedifferentiation. In one example of the maturation process, a cell, or group of cells, interacts with its cellular environment during embryogenesis and organogenesis. As maturation progresses, cells begin to form niches and these niches, or microenvironments, house stem cells that direct and regulate organogenesis. At the time of birth, maturation has progressed such that cells and appropriate cellular niches are present for the organism to function and survive post-natally. Developmental processes are highly conserved amongst the different species allowing maturation or differentiation systems from one mammalian species to be extended to other mammalian species in the laboratory. During the lifetime of an organism, the cellular composition of the organs and organs systems are exposed to a wide range of intrinsic and extrinsic factors that induce cellular or genomic damage. Ultraviolet light not only has an effect on normal skin cells but also on the skin stem cell population. Chemotherapeutic drugs used to treat cancer have a devastating effect on hematopoietic stem cells. Reactive oxygen species, which are the byproducts of cellular metabolism, are intrinsic factors that compromises the genomic integrity of the cell. In all organs or organ systems, cells are continuously being replaced from stem cell populations. However, as an organism ages, cellular damage accumulates in these stem cell populations. If the damage is inheritable, such as genomic mutations, then all progeny will be effected and thus compromised. A single stem cell clone can contribute to generations of lineages such as lymphoid and myeloid cells for more than a year and therefore have the potential to spread mutations if the stem cell is damaged. The body responds to a compromised stem cell by inducing apoptosis thereby removing it from the pool and preventing potentially dysfunctional or tumorigenic properties. Apoptosis removes compromised cells from the population, but it also decreases the number of stem cells that are available for the future. Therefore, as an organism ages, the number of stem cells decrease. In addition to the loss of the stem cell pool, there is evidence that aging decreases the efficiency of the homing mechanism of stem cells. Telomeres are the physical ends of chromosomes that contain highly conserved, tandemly repeated DNA sequences. Telomeres are involved in the replication and stability of linear DNA molecules and serve as counting mechanism in cells; with each round of cell division the length of the telomeres shortens and at a pre-determined threshold, a signal is activated to initiate cellular senescence. Stem cells and somatic cells produce telomerase, which inhibits shortening of telomeres, but their telomeres still progressively shorten during aging and cellular stress. In one teaching, or embodiment, of the invention, therapeutically reprogrammed cells, in some embodiments mesenchymal stem cells, are provided. Therapeutic reprogramming refers to a maturation process wherein a stem cell is exposed to stimulatory factors according the teachings of the present invention to yield enhanced therapeutic activity. In some embodiments, enhancement of therapeutic activity may be increase proliferation, in other embodiments, it may be enhanced chemotaxis. Other therapeutic characteristics include ability to under resistance to apoptosis, ability to overcome senescence, ability to differentiate into a variety of different cell types effectively, and ability to secrete therapeutic growth factors which enhance viability/activity, of endogenous stem cells. In order to induce therapeutic reprogramming of cells, in some cases, as disclosed herein, of umbilical cord tissue originating cells, the invention teaches the utilization of stimulatory factors, including without limitation, chemicals, biochemicals and cellular extracts to change the epigenetic programming of cells. These stimulatory factors induce, among other results, genomic methylation changes in the donor DNA. Embodiments of the present invention include methods for preparing cellular extracts from whole cells, cytoplasts, and karyplasts, although other types of cellular extracts are contemplated as being within the scope of the present invention. In a non-limiting example, the cellular extracts of the present invention are prepared from stem cells, specifically embryonic stem cells. Donor cells are incubated with the chemicals, biochemicals or cellular extracts for defined periods of time, in a non-limiting example for approximately one hour to approximately two hours, and those reprogrammed cells that express embryonic stem cell markers, such as Oct4, after a culture period are then ready for transplantation, cryopreservation or further maturation. In another embodiment of the present invention, hybrid stem cells are provided which can be used for cellular regenerative/reparative therapy. The hybrid stem cells of the present invention are pluripotent and customized for the intended recipient so that they are immunologically compatible with the recipient. Hybrid stem cells are a fusion product between a donor cell, or nucleus thereof, and a host cell. Typically the fusion occurs between a donor nucleus and an enucleated host cell. The donor cell can be any diploid cell, including but not limited to, cells from pre-embryos, embryos, fetuses and post-natal organisms. More specifically, the donor cell can be a primordial sex cell, including but not limited to, oogonium or differentiated or undifferentiated spermatogonium, or an embryonic stem cell. Other non-limiting examples of donor cells are therapeutically reprogrammed cells, embryonic stem cells, fetal stem cells and multipotent adult progenitor cells. Preferably the donor cell has the phenotype of the intended recipient. The host cell can be isolated from tissues including, but not limited to, pre-embryos, embryos, fetuses and post-natal organisms and more specifically can include, but is not limited to, embryonic stem cells, fetal stem cells, multipotent adult progenitor cells and adipose-derived stem cells. In a non-limiting example, cultured cell lines can be used as donor cells. The donor and host cells can be from the same individual or different individuals. In one embodiment of the present invention, lymphocytes are used as donor cells and a two-step method is used to purify the donor cells. After the tissues was disassociated, an adhesion step was performed to remove any possible contaminating adherent cells followed by a density gradient purification step. The majority of lymphocytes are quiescent (in G0 phase) and therefore can have a methylation status than conveys greater plasticity for reprogramming. Multipotent or pluripotent stem cells or cell lines useful as donor cells in embodiments of the present invention are functionally defined as stem cells by their ability to undergo differentiation into a variety of cell types including, but not limited to, adipogenic, neurogenic, osteogenic, chondrogenic and cardiogenic cell.

In some embodiments, host cell enucleation for the generation of hybrid stem cells according to the teachings of the present invention can be conducted using a variety of means. In a non-limiting example, ADSCs were plated onto fibronectin coated tissue culture slides and treated with cells with either cytochalasin D or cytochalasin B. After treatment, the cells can be trypsinized or the use of TryplE, re-plated and are viable for about 72 hours post enucleation. Host cells and donor nuclei can be fused using one of a number of fusion methods known to those of skill in the art, including but not limited to electrofusion, microinjection, chemical fusion or virus-based fusion, and all methods of cellular fusion are envisioned as being within the scope of the present invention. The hybrid stem cells made according to the teachings of the present invention possess surface antigens and receptors from the enucleated host cell but has a nucleus from a developmentally younger cell. Consequently, the hybrid stem cells of the present invention will be receptive to cytokines, chemokines and other cell signaling agents, yet possess a nucleus free from age-related DNA damage. The therapeutically reprogrammed cells and hybrid stem cells made in accordance with the teachings of the present invention are useful in a wide range of therapeutic applications for cellular regenerative/reparative therapy. For example, and not intended as a limitation, the therapeutically reprogrammed cells and hybrid stem cells of the present invention can be used to replenish stem cells in animals whose natural stem cells have been depleted due to age or ablation therapy such as cancer radiotherapy and chemotherapy. In another non-limiting example, the therapeutically reprogrammed cells and hybrid stem cells of the present invention are useful in organ regeneration and tissue repair. In one embodiment of the present invention, therapeutically reprogrammed cells and hybrid stem cells can be used to reinvigorate damaged muscle tissue including dystrophic muscles and muscles damaged by ischemic events such as myocardial infarcts. In another embodiment of the present invention, the therapeutically reprogrammed cells and hybrid stem cells disclosed herein can be used to ameliorate scarring in animals, including humans, following a traumatic injury or surgery. In this embodiment, the therapeutically reprogrammed cells and hybrid stem cells of the present invention are administered systemically, such as intravenously, and migrate to the site of the freshly traumatized tissue recruited by circulating cytokines secreted by the damaged cells. In another embodiment of the present invention, the therapeutically reprogrammed cells and hybrid stem cells can be administered locally to a treatment site in need or repair or regeneration.

In one embodiment, umbilical cord samples were obtained following the delivery of normal term babies with Institutional Review Board approval. A portion of the umbilical cord was then cut into approximately 3 cm long segments. The segments were then placed immediately into 25 ml of phosphate buffered saline without calcium and magnesium (PBS) and 1.times. antibiotics (100 U/ml penicillin, 100 ug/ml streptomycin, 0.025 ug/ml amphotericin B). The tubes were then brought to the lab for dissection within 6 hours. Each 3 cm umbilical cord segment was dissected longitudinally utilizing aseptic technique. The tissue was carefully undermined and the umbilical vein and both umbilical arteries were removed. The remaining segment was sutured inside out and incubated in 25 ml of PBS, 1.times. antibiotic, and 1 mg/ml of collagenase at room temperature. After 16-18 hours the remaining suture and connective tissue was removed and discarded. The cell suspension was separated equally into two tubes, the cells were washed 3.times. by diluting with PBS to yield a final volume of 50 ml per tube, and then centrifuged. Red blood cells were then lysed using a hypotonic solution. Cells were plated onto 6-well plates at a concentration of 5-20.times.10.sup.6 cells per well. UC-MSC were cultured in low-glucose DMEM (Gibco) with 10% FBS (Hyclone), 2 mM L-Glutamine (Gibco), 100 U/ml penicillin, 100 ug/ml streptomycin, 0.025 ug/ml amphotericin B (Gibco). Cells were washed 48 hours after the initial plating with PBS and given fresh media. Cell culture media were subsequently changed twice a week through half media changes. After 7 days or approximately 70-80% confluence, cells were passed using HyQTase (Hyclone) into a 10 cm plate. Cells were then regularly passed 1:2 every 7 days or upon reaching 80% confluence. Alternatively, 0.25% HQ trypsin/EDTA (Hyclone) was used to passage cells in a similar manner.

In some embodiments of the invention, administration of cells of the invention is performed for suppression of an inflammatory and/or autoimmune disease. In these situations, it may be necessary to utilize an immune suppressive/or therapeutic adjuvant. Immune suppressants are known in the art and can be selected from a group comprising of: cyclosporine, rapamycin, campath-1H, ATG, Prograf, anti IL-2r, MMF, FTY, LEA, cyclosporin A, diftitox, denileukin, levamisole, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, and trimetrexate), purine analogs (e.g., cladribine, fludarabine, 6-mercaptopurine, thiamiprine, and thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, and tegafur) fluocinolone, triaminolone, anecortave acetate, fluorometholone, medrysone, prednislone, etc. In another embodiment, the use of stem cell conditioned media may be used to potentiate an existing anti-inflammatory agent. Anti-inflammatory agents may comprise one or more agents including NSAIDs, interleukin-1 antagonists, dihydroorotate synthase inhibitors, p38 MAP kinase inhibitors, TNF-α inhibitors, TNF-α sequestration agents, and methotrexate. More specifically, anti-inflammatory agents may comprise one or more of, e.g., anti-TNF-α, lysophylline, alpha 1-antitrypsin (AAT), interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (eg., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.-acetamidocaproic acid, s-adenosylmethionine, 3-amino-4-hydroxybutyric .acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid.

An embodiment of the present disclosure provides a modified mesenchymal stem cell culture medium, comprising a basal culture medium, a first component, and melatonin, wherein the first component is at least one selected from the group consisting of coenzyme Q10 and mitoquinone mesylate, and has a working concentration in a range from 1 .mu.M to 20 .mu.M, the melatonin has a working concentration in a range from 1 .mu.M to 20 .mu.M, and the first component and the melatonin are in a molar ratio ranging from 1:0.2 to 1:10. The modified mesenchymal stem cell culture medium can delay the decrease of mitochondrial activity in mesenchymal stem cells and increase the number of mitochondria. The mesenchymal stem cells cultured in this culture medium have high mitochondrial activity and a large number of mitochondria. Specifically, the basal culture medium is used to provide essential nutrients for the growth of the mesenchymal stem cells. The basal culture medium in the present disclosure is one selected from DMEM, EMEM, IMDM, GMEM, RPMI-1640, and .alpha.-MEM. The basic culture medium may be a serum-free mesenchymal stem cell culture medium, or a serum-containing mesenchymal stem cell culture medium. In an embodiment, the modified mesenchymal stem cell culture medium contains an additive. Specifically, the additive may be well-known additives as long as they do not inhibit the proliferation of the mesenchymal stem cells. There are, for example, growth factors (such as insulin, etc.), iron sources (such as transferrin, etc.), polyamines (such as putrescine, etc.), minerals (such as sodium selenate, etc.), sugars (such as glucose, etc.), organic acids (such as pyruvic acid, lactic acid, etc.), or the like. In an embodiment, the first component is coenzyme Q10. In an embodiment, the first component is mitoquinone mesylate (mitoQ for short). In an embodiment, the working concentration of the first component is in a range from 1 .mu.M to 20 .mu.M. The first component at the working concentration in a range from 1 .mu.M to 20 .mu.M may protect the mitochondira, increase the mitochondrial activity, and reduce ROS. Further, the working concentration of the first component is in a range from 1 .mu.M to 10 .mu.M. Still further, the working concentration of the first component is in a range from 1 .mu.M to 5 .mu.M. In an embodiment, the working concentration of the coenzyme Q10 is in a range from 1 .mu.M to 20 .mu.M. The coenzyme Q10 at the working concentration in a range from 1 .mu.M to 20 .mu.M may protect the mitochondira, increase the mitochondrial activity, and reduce ROS. Further, the working concentration of the coenzyme Q10 is in a range from 1 .mu.M to 10 .mu.M. Still further, the working concentration of the coenzyme Q10 is in a range from 1 .mu.M to 5 .mu.M.

In an embodiment, the first component and the melatonin are in a molar ratio ranging from 1:0.2 to 1:10. The first component and the melatonin in a molar ratio ranging from 1:0.2 to 1:10 may promote the proliferation of mesenchymal stem cells, enhance the cell viability, increase the mitochondrial activity, and reduce ROS. Further, the first component and the melatonin are in a molar ratio ranging from 1:1 to 1:3. In an embodiment, the first component is coenzyme Q10, and the first component and the melatonin are in a molar ratio ranging from 1:0.2 to 1:10, and further, in a molar ratio ranging from 1:1 to 1:3. In an embodiment, the working concentration of the melatonin is in a range from 1 .mu.M to 20 .mu.M. Further, the working concentration of the melatonin is in a range from 1 .mu.M to 10 .mu.M. Still further, the working concentration of the melatonin Q10 is in a range from 1 .mu.M to 5 .mu.M. In an embodiment, the modified mesenchymal stem cell culture medium further comprises a second component, and the second component is at least one selected from the group consisting of nicotinamide mononucleotide (NMN), nicotinamide ribose (NR), and NADH. The second component functions together with the first component and melatonin to further increase the mitochondrial activity and the number of mitochondria in mesenchymal stem cells. Further, the first component and the second component are in a molar ratio ranging from 1:0.2 to 1:10. When the first component and the second component are in a molar ratio ranging from 1:0.2 to 1:10, the mitochondrial activity can be further increased. Still further, the first component and the second component are in a molar ratio ranging from 1:0.2 to 1:2.5. In an embodiment, the second component is the nicotinamide mononucleotide, and the nicotinamide mononucleotide has a working concentration in a range from 0.5 .mu.M to 10 .mu.M. Further, the working concentration of the nicotinamide mononucleotide is in a range from 0.5 .mu.M to 5 .mu.M. In an embodiment, the first component is coenzyme Q10, the coenzyme Q10 has a working concentration in a range from 1 .mu.M to 5 .mu.M, the working concentration of the melatonin is in a range from 1 .mu.M to 5 .mu.M, and the working concentration of the nicotinamide mononucleotide is in a range from 0.5 .mu.M to 10 .mu.M. Further, the coenzyme Q10 and the melatonin are in a molar ratio from 1:1 to 1:3; the coenzyme Q10 and the nicotinamide mononucleotide are in a molar ratio from 1:0.2 to 1:2.5. In an embodiment, the modified mesenchymal stem cell culture medium is composed of a basal culture medium, a serum substitute, EGF, BEGF, coenzyme Q10, melatonin, and nicotinamide mononucleotide; wherein the working concentration of the coenzyme Q10 is in a range from 1 .mu.M to 5 .mu.M, the working concentration of the melatonin is in a range from 1 .mu.M to 5 .mu.M, and the working concentration of the nicotinamide mononucleotide is in a range from 0.5 .mu.M to 10 .mu.M. Further, the coenzyme Q10 and the melatonin are in a molar ratio from 1:1 to 1:3; the coenzyme Q10 and the nicotinamide mononucleotide are in a molar ratio from 1:0.2 to 1:2.5. In an embodiment, the modified mesenchymal stem cell culture medium further comprises at least one of vitamin B, minerals (or inorganic salt), polyphenols, L-carnitine, alpha lipoic acid, pyrroloquinoline quinone, and creatine. It should be noted that the working concentration of the first component refers to a concentration of the first component in the modified mesenchymal stem cell culture medium; the working concentration of melatonin refers to a concentration of the melatonin in the modified mesenchymal stem cell culture medium; and the working concentration of the second component refers to a concentration of the second component in the modified mesenchymal stem cell culture medium. In an embodiment, the modified mesenchymal stem cell culture medium as described above is used to culture the bone marrow mesenchymal stem cells. In an embodiment, the modified mesenchymal stem cell culture medium as described above can also be used to culture other mesenchymal stem cells, for example, umbilical cord mesenchymal stem cells, other than the bone marrow mesenchymal stem cells, and may increase the activity and number of mitochondria of other mesenchymal stem cells. An embodiment of the present disclosure also provides a method for culturing the bone marrow mesenchymal stem cells, comprising step a) and b), specifically, step a): isolating bone marrow mesenchymal stem cells from bone marrow. Specifically, the method for isolating bone marrow mesenchymal stem cells from bone marrow is a whole bone marrow culture method (also called a direct culture method) or a density gradient centrifugation method. In an embodiment, the bone marrow mesenchymal stem cells are isolated from the bone marrow by the density gradient centrifugation method, specifically, by adding normal saline in an equal volume to the bone marrow for dilution, followed by the diluted bone marrow slowly onto a surface of lymphocyte isolation liquid (Ficoll isolation liquid) in a ratio of 1 part by volume of Ficoll isolation liquid to 2 parts by volume of the diluted bone marrow, centrifuging the resultant mixture in a horizontal centrifuge at 2000 r/min at 20.degree. C. for 25-30 minutes, and removing the bone marrow mesenchymal stem cells from the interface. Then, the bone marrow mesenchymal stem cells are washed with normal saline 2 to 3 times and counted after adding an appropriate amount of culture medium. These cells, especially when treated with hypoxia are useful for treatment of ovarian failure. In this embodiment, the bone marrow is derived from human. It is understood that in some other embodiments, the bone marrow may also be animal bone marrow, such as mouse bone marrow. In some embodiments, step a) may be omitted, and the bone marrow mesenchymal stem cells may be purchased.

In an embodiment, the step of primarily culturing the bone marrow mesenchymal stem cells includes: seeding the bone marrow mesenchymal stem cells isolated from bone marrow at a concentration of 5.times.10.sup.6 cells/mL in any of the modified mesenchymal stem cell culture medium as described above for culture, refreshing the medium after 3 days to remove non-adherent cells, and refreshing half of the spent medium every 3 to 4 days. It should be noted that the primary culture herein refers to the culture during the period from seeding the cells obtained from the tissue taken out from human's body to the first subculture. In an embodiment, a subculture step is also included after the step of primarily culturing the bone marrow mesenchymal stem cells. Specifically, the subculture step is performed when the cells reach 90% confluency. Further, the subculture step includes: digesting the bone marrow mesenchymal stem cells, and seeding the digested bone marrow mesenchymal stem cells at a density of 2.times.10.sup.5 cells/mL to 5.times.10.sup.5 cells/mL in a fresh modified mesenchymal stem cell medium for culture.

1. A method of treating ovarian failure comprising administration of a mesenchymal stem cell population generated in a manner to withstand degenerative microenvironments associated with ovarian failure.

2. The method of claim 1, wherein said degenerative microenvironments associated with ovarian failure are characterized by enhanced T cell infiltration as compared to microenvironment of a non-degenerative ovary.

3. The method of claim 1, wherein said degenerative microenvironments associated with ovarian failure are characterized by enhanced NK cell infiltration as compared to microenvironment of a non-degenerative ovary.

4. The method of claim 1, wherein said degenerative microenvironments associated with ovarian failure are characterized by enhanced gamma delta cell infiltration as compared to microenvironment of a non-degenerative ovary.

5. The method of claim 1, wherein said degenerative microenvironments associated with ovarian failure are characterized by upregulated production of interleukin-17 as compared to microenvironment of a non-degenerative ovary.

6. The method of claim 1, wherein said degenerative microenvironments associated with ovarian failure are characterized by upregulated production of interleukin-27 as compared to microenvironment of a non-degenerative ovary.

7. The method of claim 1, wherein said mesenchymal stem cell population is cultured under conditions that endow protection from microenvironment of degenerating ovary.

8. The method of claim 7, wherein said culture conditions are hypoxic conditions.

9. The method of claim 7, wherein said hypoxic conditions are conditions of reduced oxygen tension compared to normoxia.

10. The method of claim 1, wherein said cells are generated by introduction into cytoplasm of said mesenchymal stem cell extracts of cytoplasm from immune modulatory antigen presenting cells and/or defined transcription factors.

11. The method of claim 1, wherein said ability to withstand degenerative microenvironments associated with ovarian failure is ability of said mesenchymal stem cells to suppress proliferative response of a T cell in response to a proliferative signal.

12. The method of claim 11, wherein said proliferative signal is a cytokine.

13. The method of claim 11, wherein said mitogen is selected from the group consisting of: a) phytohemagglutinin; b) pokeween mitogen; and c) conconavalin A.

14. The method of claim 13, wherein said antibody crosslinks molecules selected from the group consisting of: a) CD3; b) CD2; and c) CD28.

15. The method of claim 1, wherein said composition is composed of mesenchymal stem cells, and wherein said first regenerative adjuvant is hypoxia.

16. The method of claim 1, wherein said mesenchymal stem cells possess one or more markers selected from the group consisting of: a) CD11b; b) CD11c; c) CD20; d) CD56; e) CD57 f) CD73; g) CD90; h) CD105; i) membrane bound TGF-beta; and j) neuropilin.

17. The method of claim 1, wherein said composition is capable of inhibiting T cell mediated immune responses.

18. The method of claim 17, wherein said T cell mediated immune responses comprise of Th1 cell production of cytokines.

19. The method of claim 17, wherein said cytokine is selected from the group consisting of: a) IL-2; b) IL-6; c) IL-8; d) IL-12; e) IL-15; f) IL-18; g) interferon gamma; h) TNF-alpha; and i) interleukin-33.

20. The methods of claim 1, where the mesenchymal cell is derived from an induced pluripotent stem cell (iPSc).
